(19) United States
(12) Patent Application Publication
(10) Pub. No.: US 2007/0265570 A1
(43) Pub. Date: Nov. 15, 2007

(54) Device and method for treating skin prior to performing surgery

(76) Inventor: Zachary E. Gerut, Hewlett, NY (US)

Correspondence Address: BERNARD MALINA, ESQ.;MALINA & ASSOCIATES, PLLC, Suite 501, 60 East 42nd Street, New York, NY, 10165, US

(21) Appl. No.: 11/796,635

(22) Filed: Apr. 27, 2007

Related U.S. Application Data
(60) Provisional application No. 60/795,427, filed on Apr. 27, 2006.

(51) Int. Cl. A61M 5/00

(52) U.S. Cl. 604/173

(57) ABSTRACT

A medical fluid delivery device and a method for treating skin prior to performing surgery with this device are reported herein. The device includes a manifold body having a network of communicating conduits, at least two fluid outlet ports on the body, each fitted with a coupling to each receive a hollow needle, a reservoir for delivering the medical fluid to the manifold body and at least one distance positioning support projecting from a lower surface of the body.

This application claims priority from Provisional Patent Application Ser. No. 60/795,427, filed Apr. 27, 2007, entitled “Pressure Injection Distributor.”

### BACKGROUND OF THE INVENTION

1. Field of the Invention

This invention relates to a pressure injection distributor device and a method in which the device is used for an improved plastic surgery procedure.

2. The Related Art

Surgery including, but not limited to plastic surgery such as facelifts, needs to be performed under anesthesia. Patients are rightfully concerned about the dangers of anesthesia. They worry about “going under.” Another problem with general anesthesia is that recovery times are relatively long. Swelling and bruising are further complications of surgery which need improvement. Infection is also a factor of great concern.

U.S. Pat. No. 5,052,999 (Klein) reports an improved process for administering anesthesia while performing liposuction surgery. A syringe apparatus is disclosed therein which permits the surgery to proceed under local anesthesia. Local application is safer than the general type because the patient is awake and breathing naturally without assistance. Risks of morbidity and mortality are thereby significantly reduced.

An object of the present invention is to provide a method for anesthetizing skin prior to a wide variety of surgical procedures including, but not limited to plastic surgery, particularly for facelifts, which can be conducted under local anesthesia. Another object of the present invention is to provide a method for plastic surgery which reduces recovery time, minimizes patient's surgical fears and reduces infection rates. A still further object of the present invention is to provide a device which can deliver anesthesia, antimicrobials and other medicine efficiently over a broad area subject to the surgery.

### SUMMARY OF THE INVENTION

A method for treating skin prior to performing surgery is provided which includes:

(i) preparing a device for applying a medical fluid to human skin, the device including:
(a) a manifold body having a network of communicating conduits;
(b) at least two fluid outlet ports on the body each fitted with a coupling to each receive a hollow needle;
(c) a reservoir for delivering the medical fluid to the manifold body;
(d) at least one positioning support on the body for adjusting distance between ends of the hollow needles and the skin;

(ii) positioning the device over an area of skin to be treated, ends of the hollow needles being placed in contact with the area of skin;

(iii) forcing through pressure the medical liquid from the reservoir into the manifold body and distributing the liquid evenly through the needles into the skin.

Further, a medical fluid delivery device is provided which includes:

(a) a manifold body having a network of communicating conduits;

(b) at least two fluid outlet ports on the body each fitted with a coupling to each receive a hollow needle;

(c) a reservoir for delivering the medical fluid to the manifold body;

(d) at least one positioning support on the body for adjusting distance between ends of the hollow needles and the skin.

Needles of the present invention generally are syringe needles. These have a locking structure at one end to fluid sealingly engage the coupling.

The at least two ports range in number from 2 to 10, preferably from 3 to 6. Coupling and locking structures are each preferably luer-lock receptacles.

The manifold body has an upper and a lower surface. The at least two ports project from the lower surface. In a preferred embodiment, the upper surface has a single fluid inlet port. This inlet port is fitted with an attachment portion for accepting a supply syringe or sterile tubing. Ordinarily, the attachment portion bears a male luer-lock to receive a syringe or intravenous tubing. Syringe or tubing is fluidly coupled to a reservoir such as a pressure injector or a pump injector.

Needles according to the present invention may range in diameter from about 0.00001 to about 5 mm, preferably from about 0.0001 to about 1.0 mm, more preferably from about 0.001 to about 0.1 mm. The needle at an exit end is beveled to form a sharp point for entry into skin.

Materials of construction for the manifold body may either be plastic or metallic. When plastic, the manifold is less costly and thereby disposable after a single usage. A metallic system will usually be formed of stainless steel so that it can be re-sterilized for multiple usage. Typical plastics for use in this invention include polyethylene, polypropylene, polyester or polyamide (e.g., nylon).

A spacing adjustable support system is fitted onto the manifold body. Typically, the support system involves a set of at least two, preferably four and possibly more individual supports. These supports feature legs which project from the manifold body supporting same. In the preferred embodiment, the legs at an end thereof distant from the manifold body will have a foot (i.e., a flat platform) for resting on skin. Thumbscrews are suitable for the support device. These will be screwably adjustable to modify distance from the lower surface of the manifold body to an upper surface of the treated skin. Adjustment of the support length automatically adjusts the distance that the syringe penetrates the human dermis.

Medical fluids for use in the present invention principally comprise anesthetics, analgesics, antimicrobials and inert carrier liquids. Illustrative of the anesthetics are Novocain, lidocain and epinephrine. Antimicrobials include penicillin, ampicillin and bacitracin. Carrier liquids include saline, silicones, and non-saline water.

### BRIEF DESCRIPTION OF THE DRAWING

Various objects, features and benefits of the present invention will become more readily apparent through consideration of the following drawing in which:

FIG. 1 is a schematic side view of a device according to the present invention;

FIG. 2 is a schematic top plan view of the embodiment shown in FIG. 1;

FIG. 3 is a schematic bottom plan view; and

FIG. 4 is a schematic cross-sectional view along line 4-4 of FIG. 2.

### DETAILED DESCRIPTION OF THE INVENTION

An improved device for use in treating skin prior to performing surgery including, but not limited to plastic surgery, has now been found. A key aspect is use of a manifold body to deliver anesthesia and other medical fluids to the skin undergoing surgical treatment. The manifold body allows injection by several small needles which are as fast in anesthetizing but impart much less pain than using a single larger bore needle. Larger bore needles cause more pain than utilizing 2 or 3 or more tiny needles that are close together. Roughly the same amount of medical fluid can be delivered at approximately the same rate compared to the single larger needle. Yet the advantage with the multiple smaller needles is that a person will experience much less pain.

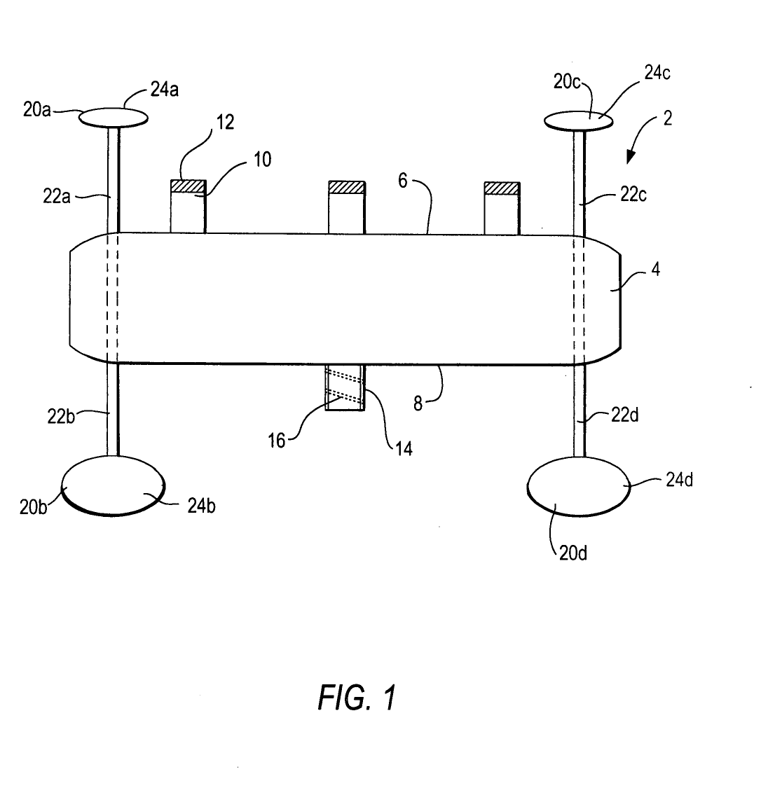

FIG. 1 illustrates a first embodiment of a medical fluid delivery device 2. The device includes a manifold body 4 having an upper surface 6 and a lower surface 8.

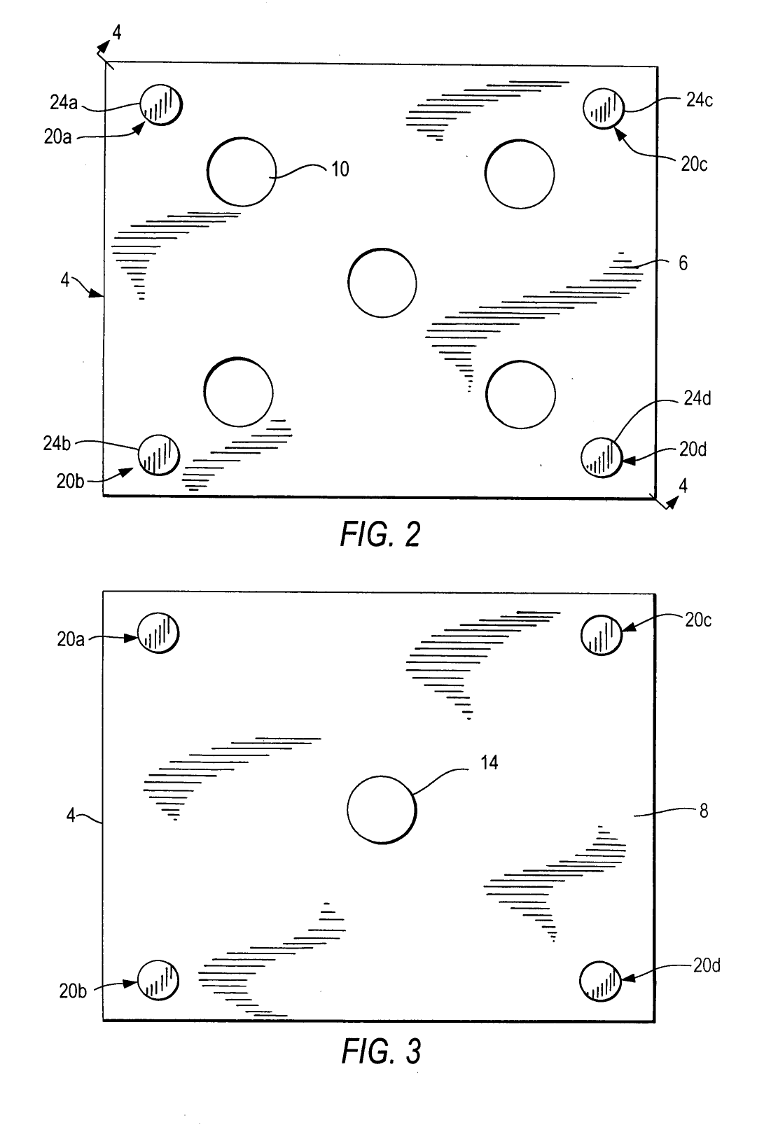

A series of fluid outlet ports 10 project from upper surface 6. FIG. 2 reveals that in the illustrated embodiment, there are 5 fluid outlet ports 10. Each of these ports are fitted with a coupling 12, and particularly a luer-type syringe coupling.

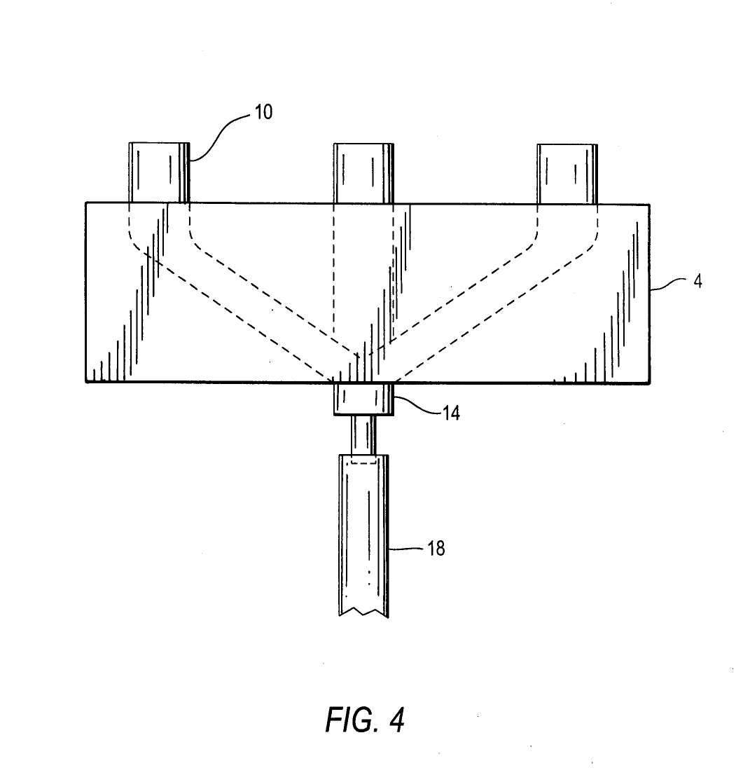

A single fluid inlet port 14 is positioned projecting outward from the upper surface (i.e., in a direction relative to skin surface) of the manifold body. Inlet port 14 is fitted with an attachment portion 16 for accepting a supply syringe or sterile tubing. A male luer lock is particularly suitable as an attachment portion. A reservoir 18 which may be in the form of a supply syringe or tubing is shown in FIG. 4 fluidly coupled to the outlet port 14.

FIGS. 2 and 3 best illustrate four distance positioning supports 20a-d which are positioned equally distant one each at the four corners of the manifold body. These supports feature a leg 22a-d and a foot 24a-d. In particular, these supports may be thumbscrews screwably adjustable to render the foot closer or further distant from the manifold body.

The manifold body need not be square or rectangular but may have any shape such as round or oval.

FIG. 4 best illustrates a series of internal conduits 26 allowing communication of medical fluid between inlet port 14 and the several outlet ports 10.

It is understood that the device of the present invention contemplates it being secured to the skin with tape, for example, and should be of sufficient weight so that it stays put. Thus, the surgeon may simply plop the device on the patient and leave it there instead of having to slowly inject novocaine.

The foregoing device is particular applicable for small procedures that normally require local anesthesia only. It is understood that the device will be used to give antibiotics in the local anesthesia.

While particular embodiments of the present invention have been described in detail, it will be apparent to those skilled in the art that the disclosed embodiments may be modified. Therefore, the foregoing description is to be considered exemplary rather than limiting, and the true scope of the invention is that defined in the following claims.

What is claimed is:

1. A medical fluid delivery device comprising:
(a) a manifold body having a network of communicating conduits;
(b) at least two fluid outlet ports on the body each fitted with a coupling to each receive a hollow needle;
(c) a reservoir for delivering the medical fluid to the manifold body; and
(d) at least one positioning support on the body for adjusting distance between ends of the hollow needles and the skin.

2. The device according to claim 1 wherein the needle is a syringe needle.

3. The device according to claim 1 wherein the needle has a locking structure to fluid sealingly engage the coupling.

4. The device according to claim 1 wherein the at least two outlet ports range in number from 2 to 10.

5. The device according to claim 1 wherein the at least two outlet ports range in number from 3 to 6.

6. The device according to claim 1 wherein the coupling is a female luer-lock.

7. The device according to claim 1 wherein the manifold body has an upper and a lower surface, the at least two inlet ports projecting from the lower surface.

8. The device according to claim 7 wherein the upper surface has a single inlet port.

9. The device according to claim 8 wherein the inlet port is fitted with an attachment portion for accepting a supply syringe or sterile tubing.

10. The device according to claim 9 wherein the supply syringe or sterile tubing serves as a reservoir.

11. The device according to claim 1 wherein each of the supports has a leg and a foot.

12. The device according to claim 1 wherein the supports are thumbscrews.

13. A method for treating skin prior to performing surgery comprising:
(i) preparing a device for applying a medical fluid to human skin, the device comprising
(a) a manifold body having a network of communicating conduits;
(b) at least two fluid outlet ports on the body each fitted with a coupling to each receive a hollow needle;
(c) a reservoir for delivering the medical fluid to the manifold body; and
(d) at least one positioning support on the body for adjusting distance between ends of the hollow needles and the skin.
(ii) positioning the device over an area of skin to be treated, ends of the hollow needles being placed in contact with the area of skin;
(iii) forcing some pressure the medical fluid from the reservoir into the manifold body and distributing the liquid evenly through the needles into the skin.
